(19) United States
(12) Patent Application Publication
(10) Pub. No.: US 2025/0135077 A1
(43) Pub. Date: May 1, 2025

(54) ELONGATED MASSAGE CUP

(71) Applicant: Mark Carangelo, East Brunswick, NJ (US)

(72) Inventor: Mark Carangelo, East Brunswick, NJ (US)

(21) Appl. No.: 18/929,631

(22) Filed: Oct. 29, 2024

Related U.S. Application Data
(60) Provisional application No. 63/546,249, filed on Oct. 30, 2023.

(51) Int. Cl. A61M 1/08

(52) U.S. Cl. CPC A61M 1/08; A61M 2205/07

(57) ABSTRACT

An elongated massage cup includes an elongated cup having an open side and a closed side. The closed side includes a valve that connects to a pump. The pump is configured to connect to the valve and evacuate air from the elongated cup when the open side of the cup is placed against a portion of a user's body, thereby creating an airtight pocket between the elongated cup and the user's body and a suction effect against the user's skin.

### CROSS-REFERENCE TO RELATED APPLICATIONS

The present application claims priority under 35 USC 119 (e) to U.S. Provisional Application No. 63/546,249 filed Oct. 30, 2023, the entire contents of which is incorporated by reference.

### FIELD OF THE INVENTION

The present invention generally relates to massage cups. Specifically, the present invention relates to an elongated massage cup.

### BACKGROUND

Cupping therapy is an ancient form of alternative medicine in which a therapist puts special cups on a cupping therapy patient's skin for a few minutes to create suction. People get cupping therapy for many reasons, including to help with pain, inflammation, blood flow, relaxation and well-being, and as a type of deep-tissue massage. The cups are generally made of: glass, bamboo, earthenware, or silicone.

Cupping therapy has recently gone mainstream and has become a popular form of treatment, but is not new. Cupping therapy or “cupping” is said to date back to ancient Egyptian, Chinese, and Middle Eastern cultures. One of the oldest medical textbooks in the world, the Ebers Papyrus, describes how the ancient Egyptians used cupping therapy in 1,550 B.C.

There are different methods of cupping, including dry cupping and wet cupping. During both types of cupping, a therapist will put a flammable substance such as alcohol, herbs, or paper in a cup and set it on fire. As the fire goes out, they put the cup upside down on a patient's skin. As the air inside the cup cools, it creates a vacuum. This causes the skin to rise and redden as blood vessels expand. The cup is generally left in place for up to 3 minutes.

A more modern version of cupping uses a rubber pump instead of fire to create the vacuum inside the cup. Sometimes therapists use silicone cups, which they can move from place to place on a person's skin for a massage-like effect.

Wet cupping creates a mild suction by leaving a cup in place for about 3 minutes. The therapist then removes the cup and uses a small scalpel to make light, tiny cuts on the patient's skin. Next, they do a second suction to draw out a small quantity of blood. Some people also get “needle cupping,” in which the therapist first inserts acupuncture needles and then puts cups over them.

A patient might get 3-5 cups in their first session. Or the patient might just try one to see how they like the therapy. The British Cupping Society notes that cupping therapy involving more than 5-7 cups is less common. Afterward, a patient may get an antibiotic ointment and bandage to prevent infection. The patient's skin should look normal again within 10 days.

Cupping therapy supporters believe that cupping therapy, particularly wet cupping, removes harmful substances and toxins from the body to promote healing. Some researchers have observed that cupping therapy may be effective when people also get other treatments, such as acupuncture or medications, for various diseases and conditions, such as: herpes zoster, acne, facial paralysis, and cervical spondylosis. The British Cupping Society says that cupping therapy may also be used to treat: blood disorders such as anemia and hemophilia, rheumatic diseases such as arthritis and fibromyalgia, fertility and gynecological disorders, skin problems such as eczema and acne, high blood pressure, migraines, anxiety and depression, bronchial congestion caused by allergies and asthma, and varicose veins.

One of the drawbacks of cupping is that the cups being used cover a small surface area of a patient's skin. As a result, cups need to be moved around to different areas or more cups are needed to cover a larger area. However, gaps between cups are left untreated and evacuating a large number of cups, either through pumping or burning, is cumbersome. There is therefore a need for a massage cup that provides more efficient treatment over a wider area of a patient's body.

### BRIEF SUMMARY

An elongated massage cup has an open side and a closed side. In one embodiment, the closed side includes a connecting port or interface that connects to a pump used to evacuate air from the massage cup. More specifically, the pump evacuates air from the elongated massage cup when the open side of the cup is pressed flush against a user's skin, creating an airtight pocket between the elongated massage cup and a portion of the user's body. This creates a suction effect against the skin of the user which causes the blood vessels under the cup to expand. The user's body then replenishes the cupped areas with healthier blood flow and stimulates proper and normal healing at a cellular level.

In addition, decompression of tissue covered by the elongated cup allows for the separation of skin, fascia and muscle to allow blood and nutrients to flow. The elongated massage cup thus increases circulation to the treated area and causes potential release of adhesions.

The elongated cup of the present invention helps eliminate clinical errors by covering a larger area of a patient's skin than traditional circular cups. Multiple elongated cups may be used to cover an even larger area of a user's body. Use of the elongated cups provide a big advantage over the traditional circle cups which leave gaps between the cups, whereas one or more elongated cups can cover an entire region. Use of the elongated cup(s) is therefore clinically critical because untreated gaps may include a clinical area of concern for the patient.

The elongated massage cup may have a variety of different shapes including semi-cylindrical, triangular, rectangular, or any other geometric shape. The open side of the elongated massage cup is defined by the cup rim which may also define or outline different shapes, such as a rectangular shaped opening. The rim may be a flanged rim that flattens out to provide better contact with the user's skin. Moreover, the rim may have a soft or cushioned cover so that pressing the open cup onto a portion of the user's body is not uncomfortable.

Although one embodiment includes an air pump for evacuating air from the elongated massage cup when pressed against a portion of the user's body, the elongated massage cup may be of the type that uses heated air caused, for example, by burning a material inside the cup and then pressing the cup against the user's skin to form a vacuum against the user's skin as the air cools. This type of elongated massage cup may not require an air pump or a port for connecting to an air pump since it uses a different principle to create a vacuum.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A shows an elongated massage cup in accordance with an embodiment of the present invention.

FIG. 1B shows an elongated massage cup in accordance with an embodiment of the present invention.

FIG. 1C shows an elongated massage cup in accordance with an embodiment of the present invention.

FIG. 1D shows an elongated massage cup in accordance with an embodiment of the present invention.

FIG. 1E shows an elongated massage cup with an air pump in accordance with an embodiment of the present invention.

FIG. 2 shows a close-up view an elongated massage cup with an air pump in accordance with an embodiment of the present invention.

FIG. 3 is a schematic view that shows a method of using the elongated massage cup in accordance with an embodiment of the present invention.

### DETAILED DESCRIPTION

Description will now be given of the invention with reference to the attached figures. It should be understood that these figures are exemplary in nature and in no way serve to limit the scope of the invention as the invention will be defined by the claims.

An elongated massage cup includes an elongated cup having an open side and a closed side. The open side of the elongated cup may be placed against a portion of a user's body to create an airtight pocket between the elongated cup and the user's skin. The closed side includes a connection port or valve that connects to a pump used to evacuate air from the elongated cup as it sits flush against a user's skin. The vacuum formed inside the elongated cup creates a suction effect that causes blood vessels under the cup to dilate and blood flow to increase around that area.

The elongated cup may have a variety of different shapes including a semi-cylindrical shape, triangular shape, rectangular shape, or any other geometric form. The shape of the elongated cup may be defined by a front profile of the elongated cup. For example, the semi-cylindrical elongated cup would have a semi-circular front profile, while the rectangular shaped elongated cup would have a rectangular front profile.

The open side of the elongated massage cup is defined by a rim of the elongated cup. The rim may define a rectangular shaped opening or any other shape. Pressing the elongated cup against the skin of a user may leave an imprint on the skin in the shape of the opening. Therefore, the rim may be flanged to create a flat contact surface between the rim and a user's skin and reduce or eliminate any imprint on the user's skin following the cupping treatment. The rim may also be covered with a soft material or cushioning, such as felt, fabric, rubber, silicone, leather, or plastic to avoid any imprint or damage to a user's skin caused by the cupping treatment.

When the open side of the elongated cup is pressed against a user's skin it forms an airtight vacuum between the elongated cup and the user's body. The air trapped in the elongated cup can then be evacuated using a pump. The pump may be connected to a port or valve on or extending from the closed side of the elongated cup. For example, a pump nozzle at the end of a hose may be connected to a valve in the elongated cup to pump air out of the cup using for example a hand operated pump. However, one of ordinary skill in the art will appreciate that any type of air pump may be used to evacuate air from the elongated cup including an electric pump.

The closed side of the elongated cup is defined by a wall of the elongated cup. The wall of the elongated cup may be curved or angled (e.g., rectangular, triangular, etc.). The connection port or valve may extend from the wall or may be flush with the wall and may be positioned anywhere along the wall but is preferably positioned at or near the middle of the elongated cup for stability and ease of access to the valve when the cup is on a user's skin.

According to another embodiment, a method of using an elongated massage cup includes: placing an open side of the elongated massage cup on a portion of a user's body forming an airtight pocket between the elongated massage cup and the user's body 302; and using a pump apparatus that connects to a valve on the elongated massage cup to evacuate air from the elongated massage cup, thereby creating a suction effect against the user's skin 304. As a result of these conditions, vasodilation occurs in the area under or near the elongated cup causing increased blood flow to that area 306.

The open side of the elongated massage cup is defined by a rim of the elongated cup. The rim may be flanged to provide a flat contact surface with the user's skin. The flanged rim helps to avoid an imprint or damage to the user's skin that may be caused by placing or pressing the elongated cup against the user's skin for an extended period of time. In addition, the rim may be covered by a soft or cushioned material for additional comfort to the user when the elongated cup is placed against the skin.

The elongated massage cup of the present invention may be made of a variety of different materials including glass, plastic, silicon, bamboo, wood, a metal such as aluminum, earthenware, or any other suitable material. In addition, the connection port or valve may be a separate attachable component or may be integrated or molded into the elongated massage cup. The valve may be an external valve extending from a surface of the massage cup or an internal valve that extends into the massage cup and has an opening flush with the outer wall of the massage cup. In either case, the valve performs the same function by interfacing with a pump to remove air from the cup.

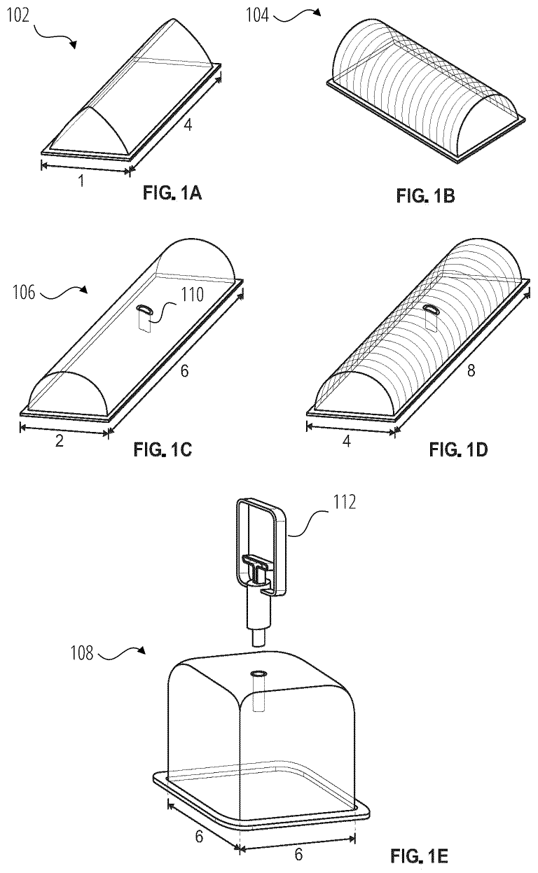

Turning to the figures, FIGS. 1A-1D show different embodiments of the elongated massage cup. FIG. 1A shows an elongated cup 102 with a triangular front profile, while FIG. 1B shows an elongated cup 104 with a semi-circular front profile. One of ordinary skill in the art will appreciate that the elongated cup can have other shapes and is not limited to any one particular shape.

The embodiments shown in FIGS. 1C and 1D show an elongated massage cup 106 with a connection port or valve 110. The valve 110 is configured to connect with an air pump used to evacuate air from inside the elongated cup when the cup is placed against a portion of a user's body. For example, a nozzle of the air pump may be configured to connect to the valve 110 of the elongated cup 106. The valve 110 allows air to be pumped out of the elongated cup, but otherwise maintains a vacuum when the cup is abutting the user's body.

FIG. 1E shows another embodiment of the elongated massage cup 108 having a rectangular shape. FIG. 1E further shows a hand pump 112 configured to connect to the connection port 110 of the elongated massage cup. More specifically, a nozzle portion at the end of a hose of the pump 112 connects to the connection port 110 which extends from the elongated cup. As already discussed, the connection port may be a valve that allows air to be pumped out of the cup when it abuts a portion of a user's body.

As illustrated in FIGS. 1A-1E, the elongated massage cup may have different shapes, and may or may not include a valve. As previously discussed, a vacuum may be created without the pump by heating the air under the massage cup and then placing it on the patient. The heated air causes vasodilation of the area under the cup along with increased blood flow to the area. As the air cools a vacuum is formed under the cup creating a suction effect. These conditions are said to have a therapeutic effect, such as lowering blood pressure, releasing toxins, and other benefits mentioned above. The embodiment that uses a pump, however, can produce the same beneficial effects without the need to heat the air inside the cup which is more labor intensive and may pose a burn risk to the patient.

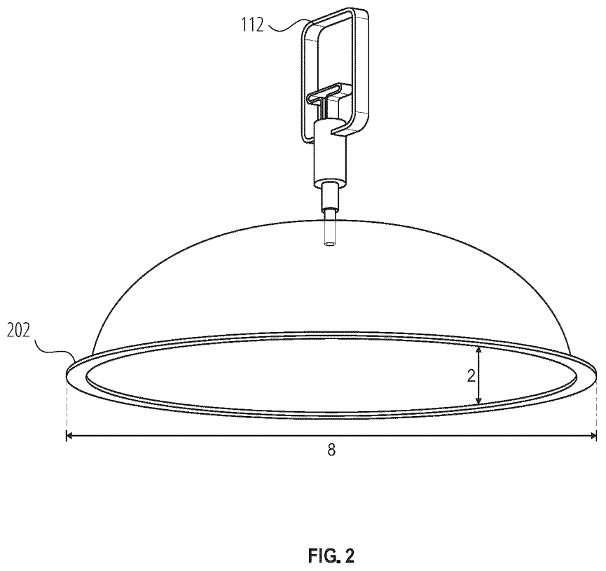

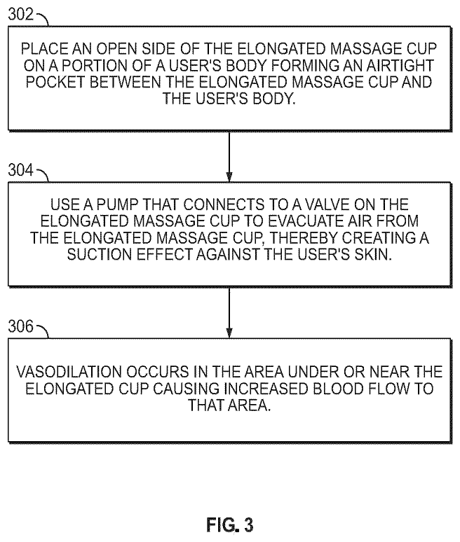

FIG. 2 shows how the pump 112 connects to the valve or connection port 110 on the back (i.e., closed side) of the elongated massage cup. FIG. 2 also illustrates the flanged rim 202 which provides a flat surface for contact with the user's skin. As can be seen, the pump 112 is a hand operated manual pump. However, one of ordinary skill in the art will appreciate that an automatic or electric pump may be used instead.

Use of the elongated cup helps to eliminate clinical errors by covering a larger area than commonly used circle cups. When treating a large area of a patient's body, circle cups leave gaps, whereas one or more elongated cups would cover the entire region, which is clinically critical since uncovered gaps may include an area of concern.

One or more elongated cups may be used to cover an entire body part. For example, the entire length of a patient's hamstrings or length of back may be covered by one or more elongated cups.

Another advantage of using elongated cup(s) compared to the smaller circular cup variety, is that the elongated cup(s) require less cleaning since there are less cups being used. Using fewer larger elongated cups also saves time because the elongated cup takes less time (approximately 3 seconds) to administer whereas the same area would require five or more smaller circular cups.

It will be understood by those of ordinary skill in the art that various changes may be made and equivalents may be substituted for elements without departing from the scope of the invention. In addition, many modifications may be made to adapt a particular feature or material to the teachings of the invention without departing from the scope thereof. Therefore, it is intended that the invention not be limited to the particular embodiments disclosed, but that the invention will include all embodiments falling within the scope of the claims.

What is claimed is:

1. An elongated massage cup comprising:
an elongated cup having an open side and a closed side, wherein the closed side includes a valve that connects to a pump;
an air pump configured to connect to the valve, wherein the pump evacuates air from the elongated cup when the open side of the cup is placed against a portion of a user's body, thereby creating an airtight pocket between the elongated cup and the user's body and a suction effect against the user's skin.

2. The elongated massage cup of claim 1, wherein the elongated cup is semi-cylindrical.

3. The elongated massage cup of claim 1, wherein the elongated cup has a semi-circular front profile.

4. The elongated massage cup of claim 1, wherein the elongated cup has a triangular front profile.

5. The elongated massage cup of claim 1, wherein the elongated cup has a rectangular front profile.

6. The elongated massage cup of claim 1, wherein the open side is defined by a rim of the elongated cup.

7. The elongated massage cup of claim 6, wherein the rim is flanged to create a flat contact surface with a user's skin when the elongated cup is pressed against a user's skin.

8. The elongated massage cup of claim 7, wherein when the open side of the elongated cup is pressed against a user's skin it forms an airtight vacuum between the elongated cup and the user's body.

9. The elongated massage cup of claim 8, wherein air in the elongated cup is evacuated using the pump.

10. The elongated massage cup of claim 1, wherein a rim of the elongated cup defines a rectangular opening.

11. The elongated massage cup of claim 1, wherein the connecting port is configured to connect to a nozzle of the pump.

12. The elongated massage cup of claim 11, wherein the nozzle is attached to a hose of the pump.

13. The elongated massage cup of claim 11, wherein the closed side is defined by a wall of the elongated cup.

14. The elongated massage cup of claim 11, wherein the valve extends from the wall.

15. A method of using an elongated massage cup comprising:
pressing an open side of the elongated massage cup to a portion of a user's body forming an airtight pocket between the elongated massage cup and the user's body;
using a pump apparatus that connects to a valve on the elongated massage cup to evacuate air from the elongated massage cup, thereby creating a suction effect against the user's skin.

16. The method of claim 15, wherein the open side is defined by a rim of the elongated massage cup.

17. The elongated massage cup of claim 16, wherein the rim defines a flanged flat surface that is placed against a user's skin.

18. The elongated massage cup of claim 17, wherein when the open side of the elongated cup is placed against a user's skin it forms an airtight vacuum.
